(19) United States
(12) Patent Application Publication
(10) Pub. No.: US 2022/0146535 A1
(43) Pub. Date: May 12, 2022

(54) COMPOUNDS AND METHODS TARGETING HUMAN TAU

(71) Applicant: Eli Lilly and Company, Indianapolis, IN (US)

(72) Inventors: Xiyun CHAI, Carmel, IN (US); Jinbiao CHEN, Carmel, IN (US); Jeffrey L. Dage, Fishers, IN (US); David Albert Driver, Solana Beach, CA (US); Steven Fisher Hinton, Escondido, CA (US); Robert William Siegel, II, Fountaintown, IN (US); Peter Edward Vaillancourt, Del Mar, CA (US)

(21) Appl. No.: 17/535,059

(22) Filed: Nov. 24, 2021

Related U.S. Application Data
(63) Continuation of application No. PCT/US2020/034274, filed on May 22, 2020.
(60) Provisional application No. 62/855,331, filed on May 31, 2019.

(51) Int. Cl. G01N 33/68; C07K 16/18

(52) U.S. Cl. CPC G01N 33/6896; C07K 16/18; C07K 2317/92; G01N 2333/4709; G01N 2800/2821

(57) ABSTRACT

The present invention provides compounds and methods targeting human tau, particularly human tau phosphorylated at threonine 217 and isoforms of tau expressed only in the CNS, including therapeutic antibodies, pharmaceutical compositions and diagnostic applications useful in the field of neurodegenerative diseases such as AD, PSP and FTD.

The present invention is in the field of medicine. More particularly, the present invention relates to compounds, pharmaceutical compositions, diagnostics and methods which include an antibody or fragment thereof directed against human tau. The compounds and methods of the present invention are expected to be useful in the field of neurodegenerative diseases, particularly tauopathies including Alzheimer's disease (AD), Progressive Supranuclear Palsy (PSP) and Frontal Temporal Dementia (FTD), and the like, including treatment thereof and diagnostics relating thereto.

Tau is an axonal microtubule binding protein, expressed in both the central nervous system (“CNS”) and peripherally, which promotes microtubule assembly and stability. Known isoforms of human tau expressed in the CNS and are involved in the aberrant formation and aggregation of intraneuronal neurofibrillary tangles (“NFTs”). In neurodegenerative diseases such as AD, the density and neuroanatomical localization of CNS NFTs correlate with severity of dementia, extent of neuronal loss and overall disease progression. In PSP, CNS NFT formation is also seen, the density of which has also been correlated with the severity of neuronal loss.

AD is a neurodegenerative disease characterized by dementia, causing problems with memory, thinking and behavior. According to the Alzheimer's Association, there are an estimated 5.6 million Americans 65 years or older (i.e., approx. 1 in 10) with AD and another 200,000 Americans under 65 with AD. The Alzheimer's Association also notes an expected increase of over 26% by 2025 in the number of 65 and older Americans with AD. This represents a significant healthcare expenditure; in 2019 alone direct AD related healthcare costs are estimated to reach 290 billion dollars in the United States and this figure does not include unpaid caregiver costs. Despite the significant personal and healthcare impact of AD, to date there are no approved disease modifying therapeutics for the treatment of AD and such treatment remains an unmet medical need.

Additionally, in order to aide in the discovery and/or development of a disease modifying treatment, reliable and sensitive diagnostics for AD are needed. An approved diagnostic applications for AD is Amyvid™. Flourtaucipir is a diagnostic application for AD currently under FDA review. Both Amyvid™ and flortaucipir are radioisotopic neurological imaging agents useful in the detection and staging of AD and other neurodegenerative diseases. Additionally, a diagnostic assay targeting the phosphorylated threonine at residue 181 of human tau (“hTau-pT181”) (residue number based on SEQ ID NO. 1) in patient samples was recently disclosed. However, the hTau-pT181 diagnostic application lacks the sensitivity necessary for diagnostic testing, such as identifying different AD stages of patients or patient prognosis, in blood, plasma and cerebrospinal fluid (“CSF”) assay. Thus, a diagnostic applicable for testing with blood, plasma and/or CSF, which offers a less expensive and less invasive diagnostic option, and which is also sensitive and reliable, is needed. Preferably, such diagnostic will be capable of identifying and/or differentiating between AD patients (for example, based on stage of AD or prognosis). Such diagnostic will also preferably be able to identify and/or differentiate between efficacious therapeutic responses. In embodiments, such diagnostic will also preferably be able to identify and/or differentiate between patients in need of further diagnostic evaluation, for example, patients for which neurological imaging, such as flourtaucipir and/or amyvid, is appropriate.

Accordingly, in an embodiment the present disclosure provides antibodies, and pharmaceutical compositions thereof, directed against human tau phosphorylated at threonine at residue 217 (residue number based on SEQ ID NO. 1) (“hTau-pT217”) as well as methods and diagnostic applications using such antibodies and pharmaceutical compositions. Additionally, according to an embodiment of the present disclosure, antibodies, and pharmaceutical compositions thereof, are provided that are directed against isoforms of human tau expressed in the CNS (e.g., recognizing the isoforms expressed in the CNS and not recognizing isoforms of human tau expressed exclusively outside the CNS).

According to some embodiments, antibodies are provided which specifically bind hTau-pT217. In more specific embodiments, antibodies are provided which bind an epitope region of human tau comprising phosphorylated threonine at residue 217 of SEQ ID NO. 1, wherein such antibodies do not bind human tau if threonine at residue 217 of SEQ ID NO. 1 is not phosphorylated. In even more specific embodiments of the present disclosure such antibodies comprise a light chain variable region (LCVR) and a heavy chain variable region (HCVR) are provided, wherein the LCVR comprises complementarity determining regions (CDRs) LCDR1, LCDR2, and LCDR3 and the HCVR comprises CDRs HCDR1, HCDR2 and HCDR3 wherein LCDR1 has the amino acid sequence of SEQ ID NO: 13, LCDR2 has the amino acid sequence of SEQ ID NO: 14, LCDR3 has the amino acid sequence of SEQ ID NO: 15, HCDR1 has the amino acid sequence of SEQ ID NO: 10, HCDR2 has the amino acid sequence of SEQ ID NO: 11, and HCDR3 has the amino acid sequence of SEQ ID NO: 12. In some embodiments, LCDR1 has an amino acid sequence with at least 95% homology to the amino acid sequence of SEQ ID NO: 13, LCDR2 has an amino acid sequence with at least 95% homology to the amino acid sequence of SEQ ID NO: 14, LCDR3 has an amino acid sequence with at least 95% homology to the amino acid sequence of SEQ ID NO: 15, HCDR1 has an amino acid sequence with at least 95% homology to the amino acid sequence of SEQ ID NO: 10, HCDR2 has an amino acid sequence with at least 95% homology to the amino acid sequence of SEQ ID NO: 11, and HCDR3 has an amino acid sequence with at least 95% homology to the amino acid sequence of SEQ ID NO: 12. According to some embodiments of antibodies provided by the present disclosure, the LCVR has the amino acid sequence of SEQ ID NO: 5 and the HCVR has the amino acid sequence of SEQ ID NO: 3. In some embodiments of antibodies provided by the present disclosure, the LCVR has the amino acid sequence of SEQ ID NO: 8 and the HCVR has the amino acid sequence of SEQ ID NO: 6. In some further embodiments, the LCVR has an amino acid sequence with at least 95% homology to the amino acid sequence of SEQ ID NO: 5 and the HCVR has an amino acid sequence with at least 95% homology to the amino acid sequence of SEQ ID NO: 3. In even further embodiments, the LCVR has an amino acid sequence with at least 95% homology to the amino acid sequence of SEQ ID NO: 8 and the HCVR has an amino acid sequence with at least 95% homology to the amino acid sequence of SEQ ID NO: 6.

According to embodiments of the present disclosure, antibodies are provided which specifically bind CNS-expressed isoforms of human tau (e.g., known isoforms of human tau expressed in the CNS) and such antibodies do not bind isoforms of human tau expressed exclusively in regions outside of the CNS, including the peripheral nervous system. According to particular embodiments, such antibodies which specifically bind CNS-expressed isoforms of human tau bind an epitope region of human tau comprising resides 124 (glutamine) and 125 (alanine) of SEQ ID NO.1. In specific embodiments, such antibodies comprise a light chain variable region (LCVR) and a heavy chain variable region (HCVR) are provided, wherein the LCVR comprises complementarity determining regions (CDRs) LCDR1, LCDR2, and LCDR3 and the HCVR comprises CDRs HCDR1, HCDR2 and HCDR3 wherein LCDR1 has the amino acid sequence of SEQ ID NO: 23, LCDR2 has the amino acid sequence of SEQ ID NO: 24, LCDR3 has the amino acid sequence of SEQ ID NO: 25, HCDR1 has the amino acid sequence of SEQ ID NO: 20, HCDR2 has the amino acid sequence of SEQ ID NO: 21, and HCDR3 has the amino acid sequence of SEQ ID NO: 22. In some embodiments, LCDR1 has an amino acid sequence with at least 95% homology to the amino acid sequence of SEQ ID NO: 23, LCDR2 has an amino acid sequence with at least 95% homology to the amino acid sequence of SEQ ID NO: 24, LCDR3 has an amino acid sequence with at least 95% homology to the amino acid sequence of SEQ ID NO: 25, HCDR1 has an amino acid sequence with at least 95% homology to the amino acid sequence of SEQ ID NO: 20, HCDR2 has an amino acid sequence with at least 95% homology to the amino acid sequence of SEQ ID NO: 21, and HCDR3 has an amino acid sequence with at least 95% homology to the amino acid sequence of SEQ ID NO: 22. According to some embodiments of antibodies provided by the present disclosure, the LCVR has the amino acid sequence of SEQ ID NO: 17 and the HCVR has the amino acid sequence of SEQ ID NO: 19. In some further embodiments, the LCVR has an amino acid sequence with at least 95% homology to the amino acid sequence of SEQ ID NO: 17 and the HCVR has an amino acid sequence with at least 95% homology to the amino acid sequence of SEQ ID NO: 19.

According to some embodiments, antibodies of the present disclosure may be humanized. In some embodiments, antibodies of the present disclosure comprise an IgG4 heavy chain. In some embodiments, antibodies of the present disclosure comprises a kappa light chain. According to even further embodiments, the present disclosure provides pharmaceutical compositions comprising an antibody of the present disclosure and one or more pharmaceutically acceptable carriers, diluents or excipients.

According to even further embodiments, the present disclosure provides a method of treating a neurodegenerative disease comprising administering to a patient in need thereof an effective amount of an antibody, or pharmaceutical composition thereof, of the present disclosure. In some such embodiments, the neurodegenerative disease is a tauopathy. In even more specific embodiments the tauopathy is one of AD, PSP and FTD.

According to some embodiments, the present disclosure provides an antibody, or pharmaceutical composition thereof, of the present disclosure for use in therapy. Additionally, an antibody, or pharmaceutical composition thereof, of the present disclosure is provided for use in the treatment of a neurodegenerative disease. In some such embodiments, the neurodegenerative disease is a tauopathy. In some more specific embodiments the tauopathy is selected from the group consisting of AD, PSP and FTD.

According to some embodiments of the present disclosure, an antibody, or pharmaceutical composition thereof, of the present disclosure is provided for use in the manufacture of a medicament for the treatment of a neurodegenerative disease. In some such embodiments, the neurodegenerative disease is a tauopathy. In even more specific embodiments the tauopathy is selected from the group consisting of AD, PSP and FTD.

According to further embodiments of the present disclosure, a method of detecting hTau-pT217 in a patient sample is provided. Such methods comprise the steps of contacting the patient sample with an antibody of the present disclosure that specifically binds hTau-pT217, and detecting a signal provided by said step of contacting.

According to embodiments, a method of detecting only CNS-expressed isoforms of human tau is provided. Such methods comprise the steps of contacting the patient sample with an antibody of the present disclosure that specifically binds isoforms of human tau expressed in the CNS (i.e., and which do not bind isoforms of human tau expressed exclusively outside of the CNS), and detecting a signal provided by said step of contacting.

According to some embodiments, a method of quantifying hTau-pT217 in a patient sample is provided. Such methods comprise the steps of contacting s patient sample with an antibody of the present disclosure that specifically binds hTau-pT217 and detecting a signal provided by said step of contacting. In some embodiments, such methods further comprise the steps of contacting a control standard with the antibody and detecting a signal provided by said step of contacting the control standard.

In some embodiments, a method of quantifying hTau-pT217 in a patient sample is provided by the present disclosure. Such methods comprise the steps of contacting the patient sample with an antibody of the present disclosure that specifically binds hTau-pT217 and contacting the patient sample with an antibody of the present disclosure that specifically bind CNS-expressed isoforms of human tau, wherein the antibodies do not bind overlapping epitope regions of the antibody and one of the antibodies comprising a detectable label; detecting a signal provided by the detectable label upon formation of a complex comprising the antibodies and hTau-pT217; contacting a control standard with the antibodies; and detecting a signal provided by the detectable label upon formation of a complex comprising the antibodies and the control standard.

According to some embodiments of the present disclosure, a method of diagnosing a patient as one or more of: (i) having a neurodegenerative disease; (ii) at risk for having a neurodegenerative disease; (iii) in need of treatment for a neurodegenerative disease; (iv) at AD Braak Stage I, II, III, IV, V or VI; or (v) in need of neurological imaging is provided. According to such embodiments, such methods comprise the steps of contacting the patient sample with an antibody of the present disclosure that specifically binds hTau-pT217 and detecting binding between the antibody and hTau-pT217 in the patient sample. In some such embodiments, the method further comprises the step of diagnosing the patent as one of: (i) having a neurodegenerative disease; (ii) at risk for having a neurodegenerative disease; (iii) in need of treatment for a neurodegenerative disease; (iv) at AD Braak Stage I, II, III, IV, V or VI; or (v) in need of neurological imaging if the level of hTau-pT217 detected in the patient sample exceeds a reference level.

In some embodiments of the present disclosure, a method of diagnosing and treating a neurodegenerative disease in a patient is provided. According to such embodiments, the methods comprise the steps of: contacting a patient sample with an antibody of the present disclosure that specifically binds hTau-pT217; detecting binding between the antibody and hTau-pT217 in the patient sample; diagnosing the patient with a neurodegenerative disease; and administering a therapeutically effective amount of an anti-human Tau antibody to the diagnosed patient. In some embodiments, the step of diagnosing comprises diagnosing the patient as having a neurodegenerative disease when the presence of hTau-pT217 in the patient sample exceeds a reference level.

According to some embodiments of the methods of the present disclosure, such methods further comprise the step of quantifying hTau-pT217 in the patient sample. In such embodiments, the step of quantifying hTau-pT217 comprises quantifying hTau-pT217 in the patient sample to a reference standard.

According to some embodiments of the methods of the present disclosure, the patient sample is one of blood, plasma, serum or CSF.

According to some embodiments of the methods of the present disclosure, the methods further comprise the step of contacting the patient sample with an antibody that specifically binds hTau-pT217 and a second antibody, said second antibody specifically binds CNS-expressed isoforms of human tau. In some such methods, one of the antibody or the second antibody comprises a detectable label and said step of detecting comprises detecting a signal provided by the detectable label upon formation of a complex comprising the antibody, the second antibody and hTau-pT217. According to some such embodiments, one of the antibody and the second antibody are immobilized on a substrate. In some embodiments of the methods of the present disclosure, the steps of contacting the patient sample with the antibody and contacting the patient sample with the second antibody occurs simultaneously. According to some more specific embodiments, the second antibody comprises an antibody of the present disclosure that specifically binds CNS-expressed isoforms of human tau as disclosed herein.

### BRIEF DESCRIPTION OF THE DRAWINGS

FIG. 1 is a ROC curve for hTau-pT217 in AD, CU-A+ and CU-A− subject groups.

As used herein, an “antibody” is an immunoglobulin molecule comprising 2 HCs and 2 LCs interconnected by disulfide bonds. The amino terminal portion of each LC and HC includes a variable region of about 100-120 amino acids primarily responsible for antigen recognition via the CDRs contained therein. The CDRs are interspersed with regions that are more conserved, termed framework regions (“FR”). Each LCVR and HCVR is composed of 3 CDRs and 4 FRs, arranged from amino-terminus to carboxy-terminus in the following order: FR1, CDR1, FR2, CDR2, FR3, CDR3, FR4. The 3 CDRs of the LC are referred to as “LCDR1, LCDR2, and LCDR3,” and the 3 CDRs of the HC are referred to as “HCDR1, HCDR2, and HCDR3.” The CDRs contain most of the residues that form specific interactions with the antigen. The functional ability of an antibody to bind a particular antigen is largely influenced by the six CDRs. Assignment of amino acids to CDR domains within the LCVR and HCVR regions of the antibodies of the present invention is based on the well-known Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)).

LCs, according to some embodiments of the present disclosure, are classified as kappa or lambda and are each characterized by a particular constant region as known in the art. HCs, according to some embodiments of the present disclosure, are classified as gamma, mu, alpha, delta, or epsilon, and define the isotype of an antibody as IgG, IgM, IgA, IgD, or IgE, respectively. According to some embodiments, the antibodies include IgG HCs, which can be further divided into subclasses, e.g., IgG1, IgG2, IgG3, IgG4. The carboxy-terminal portion of each HC defines a constant region primarily responsible for effector function. In a particular embodiment, the antibodies of the present invention have one or more modifications in the constant region of each HC that reduces effector function.

The antibodies of the present invention are monoclonal antibodies. Monoclonal antibodies are antibodies derived from a single copy or clone including, for example, any eukaryotic, prokaryotic or phage clone, and not the method by which it is produced. Monoclonal antibodies can be produced, for example, by hybridoma technologies, recombinant technologies, phage display technologies, synthetic technologies, e.g., CDR-grafting, or combinations of such or other technologies known in the art.

Methods of producing and purifying antibodies are well known in the art and can be found, for example, in Harlow and Lane (1988), Antibodies, A Laboratory Manual, Cold Spring Harbor Laboratory Press, Cold Spring harbor, N.Y., chapters 5-8 and 15, ISBN 0-87969-314-2. For example, mice or rabbits may be immunized with hTau-pT217 and the resulting antibodies can be recovered, purified, and the amino acid sequences determined using conventional methods well known in the art. Likewise, a phage library may be screened, whereby thousands of Fab fragments are screened for interaction with hTau-pT217 and resulting interactions can be recovered, purified, and the amino acid sequences determined using conventional methods well known in the art, whereby initial lead antibodies can be constructed. Embodiments of antibodies of the present disclosure include antibodies engineered to contain one or more human framework regions surrounding CDRs derived from the non-human antibody. Human framework germline sequences can be obtained, for example, from ImMunoGeneTics (INGT) via their website, http://imgt.cines.fr, or from The Immunoglobulin FactsBook by Marie-Paule Lefranc and Gerard Lefranc, Academic Press, 2001, ISBN 012441351.

In particular embodiments of the present invention, the antibody, or the nucleic acid encoding same, is provided in isolated form. As used herein, the term “isolated” refers to a protein, peptide, or nucleic acid that is free or substantially free from other macromolecular species found in a cellular environment.

Antibodies provided by the present disclosure can be used in the treatment of patients. More particularly, embodiments of antibodies of the present disclosure may be useful in the treatment of neurodegenerative diseases or disorders, including tauopathies including AD, PSP and FTD. Although antibodies of the present invention may be useful in the treatment of AD, PSP and FTD, such antibodies may also be useful in the treatment of other neurodegenerative diseases, particularly those involve tau pathology such a NFT formation. As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of an antibody of the present invention for treatment of a disease or condition in a human that would benefit from a reduction in the propagation of at least one of tau aggregate formation, NFT formation and neuronal loss, and includes: (a) inhibiting further progression of the disease, i.e., arresting its development; and (b) relieving the disease, i.e., causing regression of the disease or disorder or alleviating symptoms or complications thereof.

As used interchangeably herein, the term “patient,” “subject,” and “individual,” refers to a human. In certain embodiments, the patient is further characterized with a disease, disorder, or condition (e.g., a neurodegenerative disorder) that would benefit from a reduction in the propagation of at least one of tau aggregate formation, neurofibrillary tangles formation, and neuronal loss. In another embodiment, the patient is further characterized as being at risk of developing a neurodegenerative disorder, disease, or condition that would benefit from a reduction in the propagation of at least one of tau aggregate formation, NFT formation, and neuronal loss.

As used herein, the term “specifically binds hTau-pT217” refers to an interaction of an antibody with an epitope region of human tau comprising a phosphorylated threonine at residue 217 of SEQ ID NO. 1. Such binding is dependent upon the phosphorylation of threonine at residue 217 of SEQ ID NO. 1. It should be understood that there are known variations or isoforms of human tau, for example resulting from splice variants. It is also understood that such known variants may result in altered residue numbering for some amino acid residues of SEQ ID NO.1 including the phosphorylated threonine as is provided here in reference to the human tau sequence as set forth in SEQ ID NO. 1.

As used herein, the term “specifically binds CNS-expressed isoforms of human tau” refers to an interaction of an antibody of the present disclosure with an epitope region common to, or present on, isoforms of human tau expressed in the CNS and which epitope region is not present on isoforms of human tau expressed exclusively outside of the CNS. Antibodies which specifically bind CNS-expressed isoforms of human tau do not bind human tau isoforms expressed exclusively outside of the CNS (for example, isoforms only expressed in other regions of the body such as the peripheral nervous system). According to some embodiments, antibodies that specifically bind CNS-expressed isoforms of human tau bind to, or recognize, an epitope region of human tau isoforms expressed in the CNS comprising glutamine at residue 124 (Q124) and alanine at residue 125 (A125), residue number in reference to SEQ ID NO. 1. It should be understood that there are known variations or isoforms of human tau, for example resulting from splice variants and that such variants may result in altered residue numbering for some amino acid residues in reference to SEQ ID NO.1 including the glutamine and alanine as are provided here in reference to the human tau sequence set forth in SEQ ID NO. 1.

The term “epitope region” as used herein refers to discrete, three-dimensional sites of an antigen that are recognized, either in total or in part, by the antibodies of the present invention. The amino acids of an epitope region provide chemically active surface groupings of human tau and form a specific three-dimensional structure of human tau, and may provide specific charge characteristics. Conformational and non-conformational/linear epitopes may be distinguished in that the binding to the conformational epitope regions is lost in the presence of denaturing solvents whereas linear epitope regions is not.

An antibody of the present invention can be incorporated into a pharmaceutical composition which can be prepared by methods well known in the art and comprise an antibody of the present invention and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with an antibody of the present invention, retains the molecule's activity and is non-reactive with the patient's immune system. A pharmaceutical composition comprising an antibody of the present invention can be administered to a patient at risk for, or exhibiting, diseases or disorders as described herein by parental routes (e.g., subcutaneous, intravenous, intraperitoneal, intramuscular, or transdermal). A pharmaceutical composition of the present invention contains an “effective” or “therapeutically effective” amount, as used interchangeably herein, of an antibody of the present invention. An effective amount refers to an amount necessary (at dosages and for periods of time and for the means of administration) to achieve the desired therapeutic result. An effective amount of an antibody may vary according to factors such as the disease state, age, sex, and weight of the individual, and the ability of the antibody to elicit a desired response in the individual. An effective amount is also one in which any toxic or detrimental effects of the antibody of the present invention are outweighed by the therapeutically beneficial effects.

Percent homology, as referred to in the present disclosure, in the context of two or more amino acid sequences refers to two or more sequences having a specified percentage of amino acid residues that are the same, when compared and aligned for maximum correspondence, as measured using a sequence comparison algorithm (e.g., BLASTP and BLASTN or other algorithms available to persons of skill) or by visual inspection. Depending on the application, the percent homology can exist over a region of the sequence being compared, e.g., over a functional domain, or, alternatively, exist over the full length of the two sequences to be compared. By way of example, percent homology of a sequence may be compared to a reference sequence. For example, when using a sequence comparison algorithm, test and reference sequences may be input into a computer (and subsequence coordinates may be further designated if desired along with sequence algorithm program parameters). The sequence comparison algorithm then calculates the percent sequence identity or homology for the test sequence(s) relative to the reference sequence(s), based on the designated program parameters. Exemplary sequence alignment and/or homology algorithms are available through, Smith & Waterman, Adv. Appl. Math. 2:482 (1981), Needleman & Wunsch, J. Mol. Biol. 48:443 (1970), Pearson & Lipman, Proc. Nat'l. Acad. Sci. USA 85:2444 (1988), GAP, BESTFIT, FASTA, and TFASTA (in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

A patient “sample” as used herein refers to a human sample. Non-limiting sources of a sample for use in the present invention include blood, plasma, serum, and CSF. Additionally, sample may also refer to lymph fluid, biopsy aspirates, ascites, fluidic extracts, solid tissue, the external sections of the skin, respiratory, nasal, intestinal, and genitourinary tracts, tears, saliva, milk, tumors, organs, cell cultures and/or cell culture constituents.

The present disclosure also pertains to methods of clinical diagnosis, prognosis, or theranosis of a subject performed by a medical professional using the methods disclosed herein. The methods, as described herein, can, for example, be performed by an individual, a health professional, or a third party, for example a service provider who interprets information from the subject. As explained herein, a medical professional may initiate or modify treatment after receiving information regarding a diagnostic method of the present disclosure. For example, a medical professional may recommend a therapy, a change in therapy or an additional diagnostic assessment (e.g., a neurological imaging).

Anti-tau antibodies of the instant disclosure that specifically bind hTau-pT217 can be used to isolate, detect and/or quantify hTau-pT217 by techniques, such as affinity chromatography, immunoprecipitation, immunohistochemistry or ELISA-based assay. Such assay can be used to detect and/or evaluate the abundance and/or patterns of hTau-pT217 expression for diagnostic, prognostic, or theranostic purposes to monitor polypeptide levels, for example in serum, plasma, blood or CSF as part of a clinical testing procedure, e.g., to determine the efficacy of a given treatment regimen.

Anti-tau antibodies of the instant disclosure that specifically bind CNS-expressed isoforms of human tau can be used to isolate and/or detect isoforms of human tau expressed in the CNS (to the exclusion of isoforms of human tau expressed exclusively outside of the CNS) by techniques, such as affinity chromatography, immunoprecipitation, immunohistochemistry or ELISA-based assay. Such assay can be used to detect and/or evaluate the abundance and/or patterns of CNS-expressed isoforms of human tau expression for diagnostic, prognostic, or theranostic purposes to monitor polypeptide levels, for example in serum, plasma, blood or CSF as part of a clinical testing procedure, e.g., to determine the efficacy of a given treatment regimen. As understood in the art, an antibody of the present invention may be coupled to a detectable substance or label to facilitate its detection. Examples of detectable substances or labels include various enzymes, prosthetic groups, fluorescent materials, luminescent materials, bioluminescent materials, chemiluminescent materials and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotnazinylamine fluorescein, dansyl chloride or phycoerythnn; an example of a luminescent material includes luminol; examples of bioluminescent materials include luciferase, luciferin, ruthenium and aequorin, and examples of suitable radioactive material include ¹²⁵I, ¹³¹I, ³⁵S or ³H. Antibodies of the present invention can also be useful in pharmacogenomic analysis. Such embodiments may be used to identify individuals that can benefit from specific or modified treatment modalities and/or monitor efficacy of present treatment regimens.

Levels or measurements of hTau-pT217, as provided by assays of the present invention, may be absolute values (e.g., concentration within a biological sample) or relative values (e.g., concentration compared to a reference). As used herein, hTau-pT217 is referred to as “increased” in a patient sample if the method for detecting hTau-pT217 indicates that the level or concentration of hTau-pT217 in the patient sample is higher than a reference value. Conversely, hTau-pT217 is referred to as “decreased” in a patient sample if the hTau-pT217 level or concentration of hTau-pT217 in a patient sample is lower than a reference value, or for example, the hTau-pT217 value measured in a previous patient sample.

A “reference value” as used herein refers to a known, or approximate concentration of hTau-pT217 associated with a specific condition. The concentration levels in a reference value can be an absolute or relative amount, a range of amount, or a minimum amount, a mean amount, and/or a median amount of hTau-pT217. A reference value can also serve as a baseline of hTau-pT217 to which the patient sample is compared.

A “control standard,” as used herein, refers to a sample that can be used to compare the results obtained from a patient sample in the methods of the invention. Control standards can be cells, blood, plasma, CSF, tissue or known protein concentrations spiked into a media. The concentration levels in a control standard can be an absolute or relative amount, a range of amount, or a minimum amount, a mean amount, and/or a median amount of hTau-pT217. A control standard can also serve as a baseline of hTau-pT217 to which the patient sample is compared. The control standard can include a concentration value from the same patient or a known, normal reference of hTau-pT217. Further, in some embodiments, a control standard may express hTau-pT217 concentrations in the form of a standard curve.

As used herein, the term “capture antibody” refers to an antibody that will bind hTau-pT217. In such embodiments, the capture antibody is capable of binding and capturing hTau-pT217, for example specifically binding hTau-pT217 (e.g., not binding human Tau if the threonine at residue 217 of SEQ ID NO. 1 is not phosphorylated) in a patient sample under suitable conditions, such that the capture antibody-hTau-pT217 complex can be separated from the rest of the sample. In some embodiments, the capture antibody may be an antibody that specifically bind CNS-expressed isoforms of human tau (e.g., which may include hTau phosphorylated at the threonine at residue 217), and an antibody that specifically binds hTau-pT217 is used as the “second (or detection) antibody”. In some embodiments, the capture antibody is immobilized. In some embodiments, the detection antibody is labeled with a detectable label. In some embodiments, the capture antibody is immobilized in a “sandwich” immunoassay, and the capture or first antibody specifically binds an epitope region of human tau comprising phosphorylated threonine at residue 217 of SEQ ID NO. 1. In such sandwich immunoassays, a “detection (or second) antibody” is also utilized. According to some embodiments, a detection or second antibody may bind specifically to the capture antibody and may be labelled with a detectable label. In some embodiments, the detection of second antibody specifically binds to hTau-pT217 already bound, or captured, by the capture or first antibody. In such embodiments, the detection antibody binds hTau-pT217 at a second epitope region that does not overlap with the first or capture antibody and may be labelled with a detectable label. In some such embodiments, the second antibody is an antibody of the present invention that specifically bind CNS-expressed isoforms of human tau.

As used herein, a “detectable label” is a moiety, composition or technique that can be used to detect the formation of a complex between an antibody of the present invention that specifically binds hTau-pT217 and hTau-pT217. According to some embodiments, the detectable label may be conjugated to the antibody (either capture or detection, as the case may be) directly or indirectly. Exemplary embodiments of detectable labels include biotin; radioisotopes; fluorophores or other fluorescent moieties; and enzymatic moieties.

The term “diagnosis” or “diagnosing”, as used interchangeably herein, refers to methods by which the skilled artisan can estimate and/or determine the probability (“a likelihood”) of whether or not a patient is suffering from a given disease or condition. In the case of the present invention, “diagnosing” the patient includes using the results of an assay of the present invention to identify or diagnose a neurological disorder such as AD, PSP or FTD as well as identify a patient, for example, that is the presence or occurrence of a neurological disease or disorder or the need for treatment, or the effectiveness of a treatment against the neurological disease with the patient. A diagnosis may, according to the present invention, be based on a combination of other clinical indicia, as understood by a healthcare professional, to arrive at a diagnosis. According to some embodiments of the present invention, diagnostic applications of the present invention may be used to diagnose a patient as at an AD Braak Stage I, II, III, IV, V or VI. AD Braak stages are as know in the field and as described in Braak, et al., (2006) Acta Neuropathol 112(4): 389-404.

### EXAMPLES

### Anti-hTau-pT217 Antibodies

Anti-hTau-pT217 antibodies, or antibodies that specifically bind hTau-pT217, of the present disclosure are generated employing hybridoma methodology (e.g., as first described by Kohler et al., Nature, 256:495 (1975)). Briefly, by way of exemplification, a rabbit is immunized with a peptide including phosphorylated threonine and four or more amino acids N-terminal and C-terminal of such threonine, as represented by SEQ ID NO. 1 (an exemplification of a peptide that may be used in immunization is provided by SEQ ID NO. 26). Lymphocytes capable of producing antibodies that bind hTau-pT217 are isolated and fused with a myeloma cell line using a suitable fusing agent for forming a hybridoma cell (Goding, Monoclonal Antibodies: Principles and Practice, pp. 59-103 (Academic Press, 1986)). Hybridomas are seeded and grown in a suitable culture medium (preferably containing one or more substances inhibiting survival of unfused myeloma cells). Binding specificity of monoclonal antibodies produced by hybridomas is then determined by an in vitro binding assay (e.g., immunoprecipitation, radioimmunoassay (RIA), or enzyme-linked immunosorbent assay (ELISA)) using both hTau-pT217 and recombinant tau not phosphorylated at the threonine at residue 217 (number based on reference to SEQ ID NO. 1). Antibodies which specifically bind hTau-pT217 (e.g., and do not bind human tau not phosphorylated at residue 217, numbering based on SEQ ID NO. 1) are identified. Preferred hybridomas may be subcloned by limiting dilution procedures and grown by standard methods including in vivo as ascites tumors in an animal (Goding, Monoclonal Antibodies: Principles and Practice, pp. 59-103 (Academic Press, 1986)). Monoclonal antibodies secreted by the hybridomas (and or subclones) are purified according to conventional procedures such as, for example, affinity chromatography (e.g., protein A or protein G-Sepharose) or ion-exchange chromatography, hydroxylapatite chromatography, gel electrophoresis, dialysis, or the like.

cDNA encoding antibodies of the present invention is sequenced using conventional procedures. An exemplified rabbit anti-hTau-pT217 antibody (“mAb A”), generated following procedures substantially as described herein, comprises a heavy chain of SEQ ID NO. 2 and a light chain of SEQ ID NO. 4. Complementarity determining regions (CDRs) or variable regions of the sequenced antibodies may be used in conversion into chimeric or humanized antibodies, and/or converted to other mammalian IgG forms. For example, a clone may be converted to a murine IgG chimeric antibody such as exemplified rabbit variable region, murine IgG constant region chimeric anti-hTau-pT217 antibody (“mAb B”) having a heavy chain variable region of SEQ ID NO. 6 and a heavy chain of SEQ ID NO. 7 and a light chain variable region of SEQ ID NO. 8 and a light chain of SEQ ID NO. 9. Binding specificity may then be reassessed. cDNA sequences encoding the heavy and light chains may be cloned and engineered into a GS (glutamine synthetase) expression vector. The engineered immunoglobulin expression vector may then be stably transfected into CHO cells. As one of skill in the art will appreciate, mammalian expression of antibodies will result in glycosylation, typically at highly conserved N-glycosylation sites in the Fc region. Stable clones may be verified for expression of an antibody specifically binding to hTau-pT217. Positive clones may be expanded into serum-free culture medium for antibody production in bioreactors. Media, into which an antibody has been secreted, may be purified by conventional techniques. For example, the medium may be conveniently applied to a Protein A or G Sepharose FF column that has been equilibrated with a compatible buffer, such as phosphate buffered saline. The column is washed to remove nonspecific binding components. The bound antibody is eluted, for example, by pH gradient and antibody fractions are detected, such as by SDS-PAGE, and then pooled. The antibody may be concentrated and/or sterile filtered using common techniques. Soluble aggregate and multimers may be effectively removed by common techniques, including size exclusion, hydrophobic interaction, ion exchange, or hydroxyapatite chromatography. The product may be immediately frozen, for example at −70° C., or may be lyophilized.

Antibodies Specific for CNS-Only Expressed Isoforms of hTau

Antibodies of the present disclosure that specifically bind CNS-expressed isoforms of human tau may be generated employing hybridoma methodology (e.g., as first described by Kohler et al., Nature, 256:495 (1975)). Briefly, by way of exemplification, a non-human mammal (e.g., a murine or rabbit) may be immunized with a human tau protein (e.g., hTau given by SEQ ID NO. 1), or a peptide thereof which includes glutamine at residue 124 and alanine at residue 125, numbering as represented by SEQ ID NO. 1. Lymphocytes capable of producing antibodies that specifically bind CNS-expressed isoforms of human tau may be isolated and fused with a myeloma cell line using a suitable fusing agent for forming a hybridoma cell (Goding, Monoclonal Antibodies: Principles and Practice, pp. 59-103 (Academic Press, 1986)). Hybridomas may be seeded and grown in a suitable culture medium (preferably containing one or more substances inhibiting survival of unfused myeloma cells). Binding specificity of monoclonal antibodies produced by hybridomas is then determined by an in vitro binding assay (e.g., immunoprecipitation, radioimmunoassay (RIA), or enzyme-linked immunosorbent assay (ELISA)) against both CNS-expressed isoforms of human tau (e.g., a peptide having the sequence of SEQ ID NO. 1 and/or and peripherally expressed isoforms of human tau (e.g., a peptide having the sequence as given by SEQ ID NO. 27 which does not include the glutamine at residue 124 adjacent to the alanine at residue 125 as represented by SEQ ID NO. 1). Antibodies which specifically bind CNS-expressed isoforms of human tau (e.g., and do not bind peripherally expressed human tau) are able to be identified. Preferred hybridomas may be subcloned by limiting dilution procedures and grown by standard methods including in vivo as ascites tumors in an animal (Goding, Monoclonal Antibodies: Principles and Practice, pp. 59-103 (Academic Press, 1986)). Monoclonal antibodies secreted by the hybridomas (and or subclones) are able to be purified according to conventional procedures such as, for example, affinity chromatography (e.g., protein A or protein G-Sepharose) or ion-exchange chromatography, hydroxylapatite chromatography, gel electrophoresis, dialysis, or the like.

cDNA encoding antibodies of the present invention may be sequenced using conventional procedures. An exemplified murine antibody of the present disclosure that specifically binds CNS-expressed isoforms of human tau (“mAb C”), generated following procedures substantially as described herein, comprises a heavy chain of SEQ ID NO. 16 and a light chain of SEQ ID NO. 18. Complementarity determining regions (CDRs) or variable regions of the sequenced antibodies may be used in conversion into chimeric or humanized antibodies, and/or converted to other mammalian IgG forms and expressed in component host cells such as CHO cells.

### Binding Kinetics and Affinity

Bio-layer interferometry (BLI) assay, measured with a Octet Red96® instrument available from ForteBio (using HBS-EP+ running buffer (GE Healthcare, 10 mM Hepes pH7.4+150 mM NaCl+3 mM EDTA+0.05% surfactant P20) at 25° C.), is used to measure binding of an antibody of the present invention that binds specifically to hTau-pT217 to recombinant hTau-pT217 protein having the amino acid sequence of SEQ ID NO: 1.

Except as noted, all reagents and materials are from ForteBio (Freemont, Calif.). A Protein A biosensor is used to immobilize antibody of interest for analysis. Exemplified antibody samples of the present invention (mAb A) are prepared at 5 μg/mL by dilution into running buffer. Recombinant hTau-pT217 protein is prepared to concentrations of 300, 100, 33.3, 11.1, 3.7, 1.24, 0.4115, and 0 (blank) nM by dilution into running buffer. Each analysis consists of: (1) capturing antibody samples on biosensors for 240 secs; (2) establishing a baseline by incubating antibody loaded biosensors with running buffer for 60 secs; (3) incubating antibody loaded biosensors with serially diluted recombinant hTau-pT217 protein for 300 secs to monitor association phase; (4) return of biosensor to running buffer to monitor dissociation phase.

Binding data is processed using standard double-referencing and fit to a 1:1 binding model using Data Analysis v9.0 evaluation software to determine the association rate (kₒₙ, M⁻¹ s⁻¹ units), and dissociation rate (k_{off}, s⁻¹ units. Equilibrium dissociation constant (K_{D}) is calculated from the relationship K_{D}=k_{off}/kₒₙ, and is in molar units. Results are provided in Table 1.

TABLE 1
BLI binding data to recombinant hTau-pT217.

| Exemplified | kₒₙ | k_{off} | K_{D}* |
|---|---|---|---|
| Antibody | (M⁻¹s⁻¹ units) | (M⁻¹s⁻¹ units) | (nM) |
| mAb A | 3.39E+05 | 2.36E−04 | 6.95E−10 |

*K_{D} results are considered relative as results are not normalized for influence of avidity.

Binding Specificity to hTau-pT217

Specificity of exemplified antibodies of the present invention that specifically bind hTau-pT217 (mAb A and mAb B) is determined using synthetic peptides using a BLI assay, measured with a Octet Red96® instrument available from ForteBio (using HBS-EP+ running buffer (GE Healthcare, 10 mM Hepes pH7.4+150 mM NaCl+3 mM EDTA+0.05% surfactant P20) at 25° C.). N-terminal biotin labeled peptides of SEQ ID NO. 26, with and without phosphorylated threonine at residue 7 are immobilized to streptavidin biosensors (ForteBio). The peptides are incubated with IgG of mAb A and mAb B diluted to 5 μg/mL into running buffer for 300 secs followed by dissociation for 300 secs. Binding data is determined using Data Analysis v9.0 evaluation software. The binding signal (nm) for each peptide at the end of dissociation is provided in Table 2.

TABLE 2
BLI binding data pf mAb A and mAb B to
recombinant hTau with and without sT217.

| | phosphorylated | non-phosphorylated |
|---|---|---|
| Exemplified | threonine | threonine |
| Antibody | (nm units) | (nm units) |
| mAb A | 4.4178 | −0.0024 |
| mAb B | 4.7715 | 0.3389 |

hTau-pT217 Immunoassay

Immunoassays for measuring hTau-pT217 in plasma are designed to measure disease-related differences in AD patients. By way of exemplification, an immunoassay of the present disclosure is performed on a streptavidin small spot plate using the Meso Scale Discovery (MSD) platform. Either mAb A or mAb B are used as the capture antibody, and are biotinylated. SULFO-TAG-second antibody, such as mAb C (a antibody of the present disclosure that specifically binds CNS-expressed isoforms of human tau) is utilized as the detection antibody. Antibodies are conjugated with Sulfo-NHS-Biotin (Thermo Scientific, catalog number: 21329) or MSD GOLD SULFO-TAG NETS-Ester (MSD, catalog number: R91AO-1) according to the manufacturer's protocol. The assay is calibrated using a recombinant tau (4R2N, NCBI tau v2) protein that is phosphorylated in vitro using a reaction with glycogen synthase kinase-3 and characterized by mass spectrometry. Sample is thawed on wet ice, briefly vortexed, and plasma diluted 1:4 in Sample Buffer: for mAb A (Phosphate Buffered Saline (PBS), 0.5% Bovine serum Albumin (BSA), 0.5% Tween20, 5 mM EDTA, 5 mM EGTA); for mAb 2 50 mM HEPES, 300 mM NaCl, 5 mM EDTA, 5 mM EGTA, 1% Triton X-100, 1% MSD Blocker A, 2% PEG), with the addition of Heterophilic Blocking Reagent 1 to a concentration of 200 ug/mL (Scantibodies Inc, catalog number: 3KC533). Calibrator diluent is made by mixing the Sample Buffer 50/50 with Knock-Out Serum Replacement (Gibco, 10828-010).

MSD small-spot streptavidin (MSD, L45SA) coated plates are blocked for 1 hour at room temperature with 200 uL of 3% BSA in PBS with 650 rpm shaking on a plate shaker. The plates are washed three times with 200 uL of wash buffer (PBS+0.05% Tween 20) and 25 uL of biotinylated-capture antibody (mAb A) at 0.464 ug/mL (diluted in DPBS+0.1% BSA+0.05% Tween20, plus 2% PEG for mAb 2) added for the hTau-pT217 plates and incubated for 1 hour at room temperature with 650 rpm shaking on a plate shaker. The plates are again washed three times with 200 uL of wash buffer and 50 uL of diluted calibrator or sample added to the plate and incubated for 2 hours at room temperature with 650 rpm shaking on a plate shaker. The plates are then washed three times with 200 uL of wash buffer and 25 uL of SULFO-tagged detection antibody (mAb C) is added at 0.25 ug/mL (Diluted in MSD Diluent 35, plus 2% PEG for mAb 2) for hTau-pT217 plates and incubated for 1 hour at room temperature with 650 rpm shaking on a plate shaker. The plates are washed a final time with 200 uL of wash buffer and 150 uL of 2X MSD Read Buffer T with Surfactant (MSD, R92TC) added to each plate and read on the MSD SQ120 within 10 minutes of read buffer addition. Results are calculated by the MSD software using a 4PL, 1/y² weight of the standard curve for interpolation using the equation: Y=b₁+((b₂−b₁)/(1+(x/b₃)^{b4})).

hTau-pT217 Immunoassay as Prognostic Assay for Neurological Imaging

Levels of hTau-pT217 and hTau-pT181 are assessed in the blood of subjects enrolled in AD clinical trials. An hTau-pT217 immunoassay, as described herein, is used to assess hTau-pT217 in the plasma of AD subjects from two studies (study 1: N=42; study 2: N-185). Also, the hTau-pT181 immunoassay previously described in the field is used to measure hTau-pT181 in the same patients. All patients have tau positron emission tomography (PET) as measured by flortaucipir neurological imaging. Samples are acquired during two unique clinical trials, including at baseline, and stored at −80° C. for future biomarker research. Flortaucipir SUVR's are determined in a neocortical region of interest with respect to a reference signal in white matter. Correlations of flortaucipir SUVR and plasma pTau (pT181 and pT217, respectively) are evaluated with spearman rank-order correlations. Receiver operating characteristics (ROC) curve analyses use logistic regression models incorporating age and sex as covariates and a flortaucipir positivity cutoff of SUVR≥1.1. Baseline pTau predicting future cognitive decline is evaluated using mixed effects models evaluating pTau by quartile.

The hTau-pT217 immunoassay shows statistically significant higher correlation with Flortaucipir PET in both studies (p-value<0.05) as shown in Table 3.

TABLE 3
Correlation of hTau-pT217 and hTau-pT181 immunoassays
with Flortaucipir PET

| | hTau-pT217 | hTau-pT181 |
|---|---|---|
| | immunoassay | immunoassay |
| Study 1 | 0.783 | 0.332 |
| Study 2 | 0.463 | 0.308 |

Area under the ROC for flortaucipir positivity shows higher values for hTau-pT217 compared with hTau-pT181 in both studies (Study 1: 0.88 v. 0.79; Study 2: 0.83 v. 0.81, respectively). Additionally, mixed effects models of hTau-pT271 by quartile show significant increases of cognitive decline. As the results of the instant example demonstrates, hTau-pT217 immunoassays of the present disclosure is capable of identifying subjects suitable for neurological imaging and at risk of cognitive decline from neurodegenerative disease and shows significant improvement over hTau-pT181 immunoassay.
hTau-pT217 Immunoassay as Diagnostic Indicator for AD and Disease Progression

Levels of hTau-pT217 in CSF using an immunoassay as described are assessed and compared against assessments of hTau-pT181 immunoassay. Briefly, CSF samples unimpaired elderly (CU, n=65); patients with mild cognitive impairment due to AD (MCI-AD, n=29); AD dementia (n=43) and other neurodegenerative disorders (n=57) from the Swedish BioFINDER study are assessed with hTau-pT217 and hTau-p181 immunoassays. One hundred eighty-four participants underwent ¹⁸F-Flortaucipir positron emission tomography (PET). Uptake of ¹⁸F-Flortaucipir is quantified in a priori defined regions linked to tau pathology in AD including tau Braak stages I-II, III-IV and V-VI and inferior temp.

In CU patients, both hTau-pT217 immunoassay and hTau-pT181 immunoassay correlate with ¹⁸F-Flortaucipir at Braak stage I-II; in AD patients both hTau-pT217 immunoassay and hTau-pT181 immunoassay correlate in regions at Braak stage III-IV and V-VI; in MCI patients, hTau-pT217 correlates with ¹⁸F-Flortaucipir in regions I-II, at Braak stage III-IV and V-VI, whereas hTau-pT181 correlates only in region at Braak stage I-II. Importantly, correlations between regional ¹⁸F-Flortaucipir and hTau-pT217 immunoassay show statistically significant p<0.001-0.016) improvement over correlation between hTau-pT181 immunoassay and ¹⁸F-Flortaucipir in all three diagnostic groups (CU, MCI and AD) and across all regions (Braak stage I-II, III-IV and V-VI).

Correlation coefficients of the hTau-pT217 immunoassay show consistently higher, statistically significant (all p<0.001), values in comparison to the hTau-pT181 immunoassay for ¹⁸F-Flortaucipir across all regions: hTau-pT217 (0.698-0.752) vs. hTau-pT181 (0.572-0.706). Additionally, the hTau-pT217 immunoassay proves a statistically significant (p<0.001) more accurate predictor of pathological ¹⁸F-Flortaucipir status in all regions (hTau-pT217: AUC 0.890-0.929; hTau-pT181 immunoassay: AUC 0.859-0.904). Additionally, the hTau-pT217 immunoassay demonstrates statistically significant (p=0.026) improved performance over the hTau-pT181 immunoassay in distinguishing AD from non-AD neurodegenerative disease (hTau-pT217: AUC 0.943; hTau-pT181: AUC 0.914). These results indicate the hTau-pT217 immunoassay correlates with ¹⁸F-Flortaucipir neurological imaging and is able to differentiate AD from other neurological disorders and stages, and shows significant improvement over hTau-pT181 immunoassay.

hTau-pT217 Immunoassay Associates with Tau PET SUVr

Tau PET SUVr has been shown to be associated with Tau pathology in literature. hTau-pT217 immunoassay, as disclosed herein, correlates with Tau PET SUVr in a study measuring hTau-pT217 in plasma, serum and CSF from patients with Mild AD. Briefly, 190 subjects have hTau-pT217 immunoassay performed in plasma at baseline. Of this 190 subjects, 185 patients have Tau PET SUVr assays performed. Data is analyzed using a spearman test and a significant correlation is observed with a spearman p=0.49 and uncorrected p-value of <0.001. Additionally, 187 subjects have hTau-pT217 determined in serum at baseline. Of this 187 subjects, 182 have Tau PET SUVr assays performed. Data is analyzed using a spearman test and a significant correlation is observed with a spearman p=0.41, uncorrected p-value of <0.001. Further, 86 subjects have hTau-pT217 determined in CSF at baseline. Of this 86 subjects, 29 have Tau PET SUVr assay performed. Data is analyzed using a spearman test and a significant correlation is observed with a spearman p=0.70, uncorrected p-value<0.001. Results support the use of pTau217 levels as measured in CSF, plasma or serum to identify Tau pathology.

hTau-pT217 Immunoassay Associates with Mild AD Cognitive Status

Mean values of hTau-pT217 in plasma, serum and CSF of Mild AD subjects is calculated according to an immunoassay as described above. Results in each matrix are provided in Table 4.

TABLE 4
Mean Values of hTau-pT217 in Plasma,
Serum, and CSF of Mild AD Subjects

| Fluid | Mean | Std Dev | N |
|---|---|---|---|
| Plasma | 14.2 | 6.2 | 190 |
| Serum | 13.4 | 6.2 | 187 |
| CSF | 684.9 | 531.1 | 86 |

hTau-pT217 immunoassay, as disclosed herein, associates with cognitive status of Mild AD patients (based on Mini Mental State Exam, MMSE) and change from baseline vs. placebo for MMSE. hTau-pT217 is assessed by immunoassay in plasma, serum and CSF from patients with Mild AD. Briefly, for each of matrix (plasma, serum and CSF), subjects having hTau-pT217 measurements (as described herein) and MMSE assessment at baseline, and change from baseline assessments are evaluated for significance with spearman's test. hTau-pT217 immunoassay shows statistically significant association with both cognitive status and change from baseline in plasma and serum (CSF sample number is too low for statistical significance but data showed association such that with increased sample number, such as assessed for serum and plasma, it is expected CSF will have statistical significance associations for both MMSE and MMSE change from baseline). Results are provided in Table 5.

TABLE 5
hTau-pT217 Immunoassay Assessments in Plasma, Serum, and CSF
with MMSE and MMSE change in Baseline in Subjects Diagnosed
with Mild AD

| | | Spearman | | |
|---|---|---|---|---|
| Variable (X) | Variable (Y) | ρ | Prob>\|ρ\| | N |
| MMSE Baseline Score | Log10 CSF* | −0.16 | 1.71E−01 | 78 |
| | pTau217 | | | |
| MMSE Change from | Log10 CSF* | −0.25 | 1.48E−01 | 35 |
| Baseline (Placebo Only) | pTau217 | | | |
| MMSE Baseline Score | Log10 Plasma | −0.19 | 9.41E−03 | 187 |
| | pTau217 | | | |
| MMSE Change from | Log10 Plasma | −0.43 | 3.27E−05 | 89 |
| Baseline (Placebo Only) | pTau217 | | | |
| MMSE Baseline Score | Log10 Serum | −0.17 | 2.17E−02 | 184 |
| | pTau217 | | | |
| MMSE Change from | Log10 Serum | −0.32 | 3.11E−03 | 86 |
| Baseline (Placebo Only) | pTau217 | | | |

*not considered statistically significant due to small sample size of patients assessed for MMSE change from baseline.

These results demonstrate a cross sectional association of hTau-pT217 with a measure of cognition, MMSE and demonstrate the utility of hTau-pT217 to determine future risk of cognitive decline.

hTau-pT217 Immunoassay Associates with Amyloid Status

hTau-pT217 immunoassay, as disclosed herein, associates with amyloid status. Briefly, plasma samples from four distinct patient groups of known AD and amyloid status (based on PET neurological imagining) are assessed for hTau-pT217 association: (i) unaffected elderly amyloid positive (CU-A+); (ii) unaffected elderly amyloid negative (CU-A−); (iii) elderly amyloid positive AD (AD-A+); and (iv) clinically unaffected young (CUY-A−). Samples from each group are analyzed with hTau-pT217 immunoassay as described herein. Results are provided in Table 6.

TABLE 6
hTau-pT217 Associated with AD and Amyloid Status

| Group | Mean | Std Dev | N |
|---|---|---|---|
| Alzheimer's disease—Amyloid Positive | 11.4 | 6.9 | 14 |
| Clinically Unaffected Elderly—Amyloid Positive | 6.7 | 1.9 | 14 |
| Clinically Unaffected Elderly—Amyloid Negative | 3.6 | 1.4 | 16 |
| Clinically Unaffected Young—Amyloid Negative | 3.6 | 1.3 | 10 |

Assessment of the hTau-pT217 immunoassay for identification of amyloid positive subjects is determined by assessment of results provided in Table 6 for separate groups using a student t-test. Results are provided in Table 7.

TABLE 7
Students t-test of Mean Difference of Plasma hTau-pT217 Associated
with AD and Amyloid Status

| | | | Std | |
|---|---|---|---|---|
| | | | Err | |
| Level 1 | Level 2 | Difference | Dif | p-Value |
| Alzheimer's | Clinically Unaffected | 7.8 | 1.4 | 4.60E−06 |
| disease—Amyloid | Elderly—Amyloid | | | |
| Positive | Negative | | | |
| Alzheimer's | Clinically Unaffected | 7.8 | 1.6 | 4.67E−05 |
| disease—Amyloid | Young—Amyloid | | | |
| Positive | Negative | | | |
| Alzheimer's | Clinically Unaffected | 4.7 | 1.4 | 1.05E−02 |
| disease—Amyloid | Elderly—Amyloid | | | |
| Positive | Positive | | | |
| Clinically Unaffected | Clinically Unaffected | 3.2 | 1.4 | 1.17E−01 |
| Elderly—Amyloid | Elderly—Amyloid | | | |
| Positive | Negative | | | |

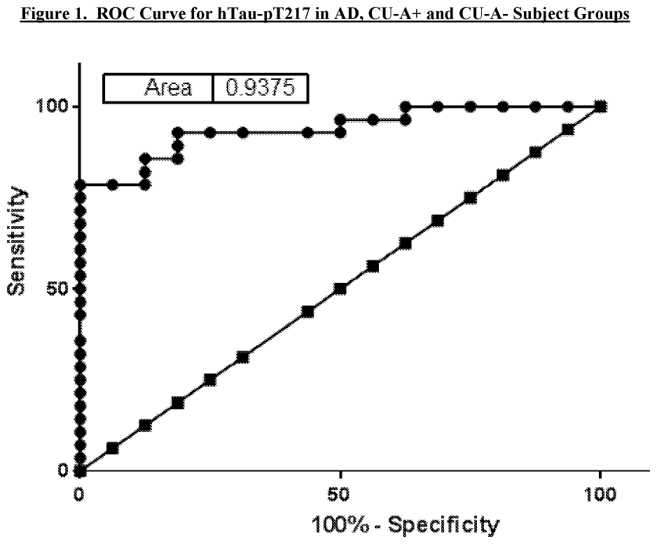

As Tables 6 and 7 show, the mean for AD-A+ group is 11.4 pg/mL and is three fold higher than age matched CU-A− group at 3.6 resulting in an uncorrected p-value of 4.60E-06. A receiver operating characteristics (ROC) curve analysis is also used to evaluate sensitivity and specificity for hTau-pT217 in the identification of amyloid positive subjects. The area under the ROC curve is 0.94 as presented in FIG. 1.

The data provided herein demonstrates the hTau-pT217 assays of the present disclosure are capable of identification of amyloid positive subjects; serving as a diagnostic for AD and determining cognitive status of subjects in relation to AD; identifying subjects at risk of AD and/or at the earliest stages of the AD; and serving as a diagnostic of progression of AD. The data also demonstrates the hTau-pT217 assays of the present disclosure correlates to neurological imaging, is functional in serum, plasma and CSF matrices and is superior to the known hTau-pT181 assay.

### Exemplified Embodiments of the Present Disclosure

1. An antibody which specifically binds human tau phosphorylated at threonine at residue 217 of SEQ ID NO. 1 (“hTau-pT217”).
2. An antibody comprising a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3,
   wherein LCDR1 has the amino acid sequence of SEQ ID NO: 13; LCDR2 has the amino acid sequence of SEQ ID NO: 14, LCDR3 has the amino acid sequence of SEQ ID NO: 15, HCDR1 has the amino acid sequence of SEQ ID NO: 10, HCDR2 has the amino acid sequence of SEQ ID NO: 11, and HCDR3 has the amino acid sequence of SEQ ID NO: 12.
3. An antibody comprising a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3,
   wherein LCDR1 has an amino acid sequence with at least 95% homology to the amino acid sequence of SEQ ID NO: 13; LCDR2 has an amino acid sequence with at least 95% homology to the amino acid sequence of SEQ ID NO: 14, LCDR3 has an amino acid sequence with at least 95% homology to the amino acid sequence of SEQ ID NO: 15, HCDR1 has an amino acid sequence with at least 95% homology to the amino acid sequence of SEQ ID NO: 10, HCDR2 has an amino acid sequence with at least 95% homology to the amino acid sequence of SEQ ID NO: 11, and HCDR3 has an amino acid sequence with at least 95% homology to the amino acid sequence of SEQ ID NO: 12.
4. The antibody of Embodiments 2 or 3, comprising a light chain variable region (LCVR) and a heavy chain variable region (HCVR) selected from:
   a. the LCVR having the amino acid sequence of SEQ ID NO: 5 and the HCVR having the amino acid sequence of SEQ ID NO: 3; and
   b. the LCVR having the amino acid sequence of SEQ ID NO: 8 and the HCVR having the amino acid sequence of SEQ ID NO: 6.
5. The antibody of Embodiments 2 or 3, comprising a light chain variable region (LCVR) and a heavy chain variable region (HCVR) selected from:
   a. the LCVR having an amino acid sequence with at least 95% homology to the amino acid sequence of SEQ ID NO: 5 and the HCVR having an amino acid sequence with at least 95% homology to the amino acid sequence of SEQ ID NO: 3; and
   b. the LCVR having an amino acid sequence with at least 95% homology to the amino acid sequence of SEQ ID NO: 8 and the HCVR having an amino acid sequence with at least 95% homology to the amino acid sequence of SEQ ID NO: 6.
6. The antibody of any of Embodiments 1-5, wherein said antibody is humanized.
7. The antibody of any of Embodiments 1-6, wherein said antibody comprises an IgG4 heavy chain.
8. The antibody of any of Embodiments 1-7, wherein said antibody comprises a kappa light chain.
9. A pharmaceutical composition comprising an antibody of any one of Embodiments 1-8 and one or more pharmaceutically acceptable carriers, diluents or excipients.
10. An antibody which specifically binds CNS-expressed isoforms of human tau.
11. The antibody of Embodiments 10, wherein the antibody binds an epitope region of human tau comprising glutamine at residue 124 and alanine at residue 125 of SEQ ID NO. 1.
12. An antibody comprising a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3,
   wherein LCDR1 has the amino acid sequence of SEQ ID NO: 23, LCDR2 has the amino acid sequence of SEQ ID NO: 24, LCDR3 has the amino acid sequence of SEQ ID NO: 25, HCDR1 has the amino acid sequence of SEQ ID NO: 20, HCDR2 has the amino acid sequence of SEQ ID NO: 21, and HCDR3 has the amino acid sequence of SEQ ID NO: 22.
13. An antibody comprising a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3,
   wherein LCDR1 has an amino acid sequence with at least 95% homology to the amino acid sequence of SEQ ID NO: 23; LCDR2 has an amino acid sequence with at least 95% homology to the amino acid sequence of SEQ ID NO: 24, LCDR3 has an amino acid sequence with at least 95% homology to the amino acid sequence of SEQ ID NO: 25, HCDR1 has an amino acid sequence with at least 95% homology to the amino acid sequence of SEQ ID NO: 20, HCDR2 has an amino acid sequence with at least 95% homology to the amino acid sequence of SEQ ID NO: 21, and HCDR3 has an amino acid sequence with at least 95% homology to the amino acid sequence of SEQ ID NO: 22.
14. The antibody of Embodiments 12 or 13, comprising a light chain variable region (LCVR) having the amino acid sequence of SEQ ID NO: 17 and a heavy chain variable region (HCVR) having the amino acid sequence of SEQ ID NO: 19.
15. The antibody of Embodiments 12 or 13, comprising a light chain variable region (LCVR) having an amino acid sequence with at least 95% homology to the amino acid sequence of SEQ ID NO: 17 and a heavy chain variable region (HCVR) having an amino acid sequence with at least 95% homology to the amino acid sequence of SEQ ID NO: 19.
16. The antibody of any of Embodiments 10-15, wherein said antibody is humanized.
17. The antibody of any of Embodiments 10-16, wherein said antibody comprises an IgG4 heavy chain.
18. The antibody of any of Embodiments 10-17, wherein said antibody comprises a kappa light chain.
19. A pharmaceutical composition comprising an antibody of any one of Embodiments 10-18 and one or more pharmaceutically acceptable carriers, diluents or excipients.
20. A method of treating a neurodegenerative disease comprising administering to a patient in need thereof an effective amount of an antibody, or pharmaceutical composition thereof, of any one of Embodiments 1-19.
21. The method of Embodiment 20, wherein the neurodegenerative disease is a tauopathy.
22. The method of Embodiment 21, wherein the tauopathy is one of AD, PSP and FTD.
23. An antibody, or pharmaceutical composition thereof, of any one of Embodiments 1-19 for use in therapy.
24. An antibody, or pharmaceutical composition thereof, of any one of Embodiments 1-19 for use in the treatment of a neurodegenerative disease.
25. The antibody, or pharmaceutical composition thereof, of Embodiment 24, wherein said neurodegenerative disease is a tauopathy.
26. The antibody, or pharmaceutical composition thereof, of Embodiment 25, wherein said tauopathy is selected from the group consisting of AD, PSP and FTD.
27. An antibody, or pharmaceutical composition thereof, of any one of Embodiments 1-19 for use in the manufacture of a medicament for the treatment of a neurodegenerative disease.
28. The antibody, or pharmaceutical composition thereof, of Embodiment 27, wherein said neurodegenerative disease is a tauopathy.
29. The antibody, or pharmaceutical composition thereof, of Embodiment 28, wherein said tauopathy is selected from the group consisting of AD, PSP and FTD.
30. A method of detecting hTau-pT217 in a patient sample comprising the steps of:
   contacting the patient sample with an antibody of any one of Embodiments 1-8; and detecting a signal provided by said step of contacting.
31. A method of quantifying hTau-pT217 in a patient sample comprising the steps of:
   contacting the patient sample with an antibody of any one of Embodiments 1-8; and detecting a signal provided by said step of contacting.
32. The method of Embodiment 31 further comprising the steps of: contacting a control standard with the antibody; and detecting a signal provided by said step of contacting the control standard.
33. A method of quantifying hTau-pT217 in a patient sample comprising the steps of:
   contacting the patient sample with an antibody of Embodiments 1-8; contacting the patient sample with a second antibody, wherein the second antibody is an antibody of Embodiments 10-18 and one of the antibody and the second antibody comprising a detectable label; detecting a signal provided by the detectable label upon formation of a complex comprising the antibody, the second antibody and hTau-pT217; contacting a control standard with the antibody; contacting the control standard with the second antibody, one of the antibody and the second antibody comprising a detectable label; and detecting a signal provided by the detectable label upon formation of a complex comprising the antibody, the second antibody and the control standard.
34. A method of diagnosing a patient as one or more of: (i) having a neurodegenerative disease; (ii) at risk for having a neurodegenerative disease; (iii) in need of treatment for a neurodegenerative disease; or (iv) in need of neurological imaging comprising the steps of: contacting the patient sample with an antibody of any one of Embodiments 1-8; and detecting binding between the antibody and hTau-pT217 in the patient sample.
35. The method of Embodiment 34 further comprising the step of diagnosing the patent as one of: (i) having a neurodegenerative disease; (ii) at risk for having a neurodegenerative disease; (iii) in need of treatment for a neurodegenerative disease; or (iv) in need of neurological imaging if the level of hTau-pT217 detected in the patient sample exceeds a reference level.
36. A method of diagnosing and treating a neurodegenerative disease in a patient, said method comprising the steps of: contacting a patient sample with an antibody of any one of Embodiment 1-8; detecting binding between the antibody and hTau-pT217 in the patient sample; diagnosing the patient with a neurodegenerative disease; and administering a therapeutically effective amount of an anti-human Tau antibody to the diagnosed patient.
37. The method of Embodiment 36, wherein said step of diagnosing comprises diagnosing the patient as having a neurodegenerative disease when the presence of hTau-pT217 in the patient sample exceeds a reference level.
38. The method of any of Embodiments 31-37 further comprising the step of quantifying hTau-pT217 in the patient sample.
39. The method of Embodiment 38, wherein said step of quantifying hTau-pT217 comprises quantifying hTau-pT217 in the patient sample to a reference standard.
40. The method of any of Embodiments 30-39, wherein the patient sample is one of blood, plasma, serum or CSF.
41. The method of any of Embodiments 30-32 and 34-40 further comprising the step of contacting the patient sample with a second antibody, said second antibody binding an epitope region of hTau-pT217 that does not overlap with the antibody.
42. The method of Embodiment 41, wherein one of the antibody or the second antibody comprises a detectable label and said step of detecting comprises detecting a signal provided by the detectable label upon formation of a complex comprising the antibody, the second antibody and hTau-pT217.
43. The method of any of Embodiments 41-42, wherein one of the antibody and the second antibody are immobilized on a substrate.
44. The method of any of Embodiments 30-43, wherein said steps of contacting the patient sample with the antibody and contacting the patient sample with the second antibody occurs simultaneously.
45. The method of any of Embodiments 32 and 41-44, wherein the second antibody comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2, and HCDR3,
   wherein LCDR1 has the amino acid sequence of SEQ ID NO: 23, LCDR2 has the amino acid sequence of SEQ ID NO: 24, LCDR3 has the amino acid sequence of SEQ ID NO: 25, and wherein HCDR1 has the amino acid sequence of SEQ ID NO: 20, HCDR2 has the amino acid sequence of SEQ ID NO: 21, and HCDR3 has the amino acid sequence of SEQ ID NO: 22.
46. The method of any of Embodiments 33 and 41-44, wherein the second antibody comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2, and HCDR3,
   wherein LCDR1 has an amino acid sequence with at least 95% homology to the amino acid sequence of SEQ ID NO: 23, LCDR2 has an amino acid sequence with at least 95% homology to the amino acid sequence of SEQ ID NO: 24, LCDR3 has an amino acid sequence with at least 95% homology to the amino acid sequence of SEQ ID NO: 25, HCDR1 has an amino acid sequence with at least 95% homology to the amino acid sequence of SEQ ID NO: 20, HCDR2 has an amino acid sequence with at least 95% homology to the amino acid sequence of SEQ ID NO: 21, and HCDR3 has an amino acid sequence with at least 95% homology to the amino acid sequence of SEQ ID NO: 22.
47. The method of any of Embodiments 46-47, wherein the second antibody comprises a light chain variable region (LCVR) and a heavy chain variable region (HCVR), the LCVR having the amino acid sequence of SEQ ID NO: 19 and the HCVR having the amino acid sequence of SEQ ID NO: 17.
48. The method of any of Embodiments 46-47, wherein the second antibody comprises a light chain variable region (LCVR) and a heavy chain variable region (HCVR), wherein the LCVR has an amino acid sequence with at least 95% homology to the amino acid sequence of SEQ ID NO: 19 and the HCVR having an amino acid sequence with at least 95% homology to the amino acid sequence of SEQ ID NO: 17.
49. A method of forming a complex between a first antibody, a second antibody and human tau expressed in the CNS and phosphorylated at threonine of residue 217 of SEQ ID NO. 1, said method comprising: contacting a patient sample with a first antibody, wherein the first antibody is an antibody of one of Embodiments 1-8; and contacting the patient sample with a second antibody, wherein the second antibody is an antibody of one of Embodiments 10-18.
50. An assay for detecting human tau expressed in the CNS and phosphorylated at threonine of residue 217 of SEQ ID NO. 1, said assay comprising: an antibody of one of Embodiments 1-8; and an antibody of one of Embodiments 10-18.
51. The assay of Embodiment 50, wherein one of the antibodies comprises a detectable label.

Sequence Listing
(hTau-pT217)
SEQ ID NO: 1
MAEPRQEFEVMEDHAGTYGLGDRKDQGGYTMHQDQEGDTDAGLKESPLQ
TPTEDGSEEPGSETSDAKSTPTAEDVTAPLVDEGAPGKQAAAQPHTEIP
EGTTAEEAGIGDTPSLEDEAAGHVTQARMVSKSKDGTGSDDKKAKGADG
KTKIATPRGAAPPGQKGQANATRIPAKTPPAPKTPPSSGEPPKSGDRSG
YSSPGSPGTPGSRSRTPSLPTPPTREPKKVAVVRTPPKSPSSAKSRLQT
APVPMPDLKNVKSKIGSTENLKHQPGGGKVQIINKKLDLSNVQSKCGSK
DNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAEI
VYKSPVVSGDTSPRHLSNVSSTGSIDMVDSPQLATLADEVSASLAKQGL
(HC of exemplified rabbit anti-hTau-pT217 antibody)
SEQ ID NO: 2
QSVEESGGRLVTPGTPLTLTCTVSGLSPSWYGVHWVRQAPGKGLEWIGV
LRAGSHTYYAGWAKGRFAISKTSTTVALKITSPTTEDTAIYFCGSVGRG
IWGPGTLVTVSLGQPKAPSVFPLAPCCGDTPSSTVTLGCLVKGYLPEPV
TVTWNSGTLTNGVRTFPSVRQSSGLYSLSSVVSVTSSSQPVTCNVAHPA
TNTKVDKTVAPSTCSKPTCPPPELLGGPSVFIFPPKPKDTLMISRTPEV
TCVVVDVSQDDPEVQFTWYINNEQVRTARPPLREQQFNSTIRVVSTLPI
AHQDWLRGKEFKCKVHNKALPAPIEKTISKARGQPLEPKVYTMGPPREE
LSSRSVSLTCMINGFYPSDISVEWEKNGKAEDNYKTTPAVLDSDGSYFL
YSKLSVPTSEWQRGDVFTCSVMHEALHNHYTQKSISRSPGK
(HCVR of exemplified rabbit anti-hTau-pT217
antibody)
SEQ ID NO: 3
QSVEESGGRLVTPGTPLTLTCTVSGLSPSWYGVHWVRQAPGKGLEWIGV
LRAGSHTYYAGWAKGRFAISKTSTTVALKITSPTTEDTAIYFCGSVGRG
IWGPGTLVTVSL
(LC of exemplified rabbit anti-hTau-pT217
antibody)
SEQ ID NO: 4
AQVLTQTASPVSATVGGTVTINCQASLAVYNNNYLAWYQQKPGQPPKRL
IYLASSLSSGVSSHFKGSGSGTQFTLTISDVQADDAATYFCQGSYDCTI
ADCVAFGGGTEVVVKGDPVAPTVLIFPPAADQVATGTVTIVCVANKYFP
DVTVTWEVDGTTQTTGIENSKTPQNSADNTYNLSSTLTLTSTQYNSHKE
YTCKVTQGTTSVVQSFNRGDC
(LCVR of exemplified rabbit anti-hTau-pT217
antibody)
SEQ ID NO: 5
AQVLTQTASPVSATVGGTVTINCQASLAVYNNNYLAWYQQKPGQPPKRL
IYLASSLSSGVSSHFKGSGSGTQFTLTISDVQADDAATYFCQGSYDCTI
ADCVAFGGGTEVVVK
(HCVR of exemplified chimeric anti-hTau-pT217
antibody)
SEQ ID NO: 6
QSVEESGGRLVTPGTPLTLTCTVSGLSPSWYGVHWVRQAPGKGLEWIGV
LRAGSHTYYAGWAKGRFAISKTSTTVALKITSPTTEDTAIYFCGSVGRG
IWGPGTLVTVSL
(HC of exemplified chimeric anti-hTau-pT217
antibody)
SEQ ID NO: 7
QSVEESGGRLVTPGTPLTLTCTVSGLSPSWYGVHWVRQAPGKGLEWIGV
LRAGSHTYYAGWAKGRFAISKTSTTVALKITSPTTEDTAIYFCGSVGRG
IWGPGTLVTVSLAKTTPPSVYPLAPGSAAQTNSMVTLGCLVKGYFPEPV
TVTWNSGSLSSGVHTFPAVLQSDLYTLSSSVTVPSSTWPSETVTCNVAH
PASSTKVDKKIVPRDCGCKPCICTVPEVSSVFIFPPKPKDVLTITLTPK
VTCVVVDISKDDPEVQFSWFVDDVEVHTAQTQPREEQFNSTFRSVSELP
IMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYF
VYSKLNVQKSNWEAGNTFTCSVLHEGLHNHHTEKSLSHSPGK
(LCVR of exemplified chimeric anti-hTau-pT217
antibody)
SEQ ID NO: 8
AQVLTQTASPVSATVGGTVTINCQASLAVYNNNYLAWYQQKPGQPPKRL
IYLASSLSSGVSSHFKGSGSGTQFTLTISDVQADDAATYFCQGSYDCTI
ADCVAFGGGTEVVVK
(LC of exemplified chimeric anti-hTau-pT217
antibody)
SEQ ID NO: 9
AQVLTQTASPVSATVGGTVTINCQASLAVYNNNYLAWYQQKPGQPPKRL
IYLASSLSSGVSSHFKGSGSGTQFTLTISDVQADDAATYFCQGSYDCTI
ADCVAFGGGTEVVVKRADAAPTVSIFPPSSEQLTSGGASVVCFLNNFYP
KDINVKWKIDGSERQNGVLNSWTDQDSKDSTYSMSSTLTLTKDEYERHN
SYTCEATHKTSTSPIVKSFNRNEC
(exemplified HCDR1)
SEQ ID NO: 10
GLSPSWYGVH
(exemplified HCDR2)
SEQ ID NO: 11
VLRAGSHTYYAGWAKG
(exemplified HCDR3)
SEQ ID NO: 12
VGRGI
(exemplified LCDR1)
SEQ ID NO: 13
QASLAVYNNNYLA
(exemplified LCDR2)
SEQ ID NO: 14
LASSLSS
(exemplified LCDR3)
SEQ ID NO: 15
LASSLSS
(HC of exemplified CNS-only expressed human tau
binding antibody)
SEQ ID NO: 16
QVQLQQWGAGLLKPSETLSLTCAVYGGSFSPYYWSWIRQPPDKGLEWIG
EINWSGDTNYNPSLKSRVTISLDTSKNQFSLNLSSVTAADTAVYYCARS
FDRWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCN
VDHKPSNTKVDKRVEPRGPTIKPCPPCKCPAPNLLGGPSVFIFPPKIKD
VLMISLSPIVTCVVVDVSEDDPDVQISWFVNNVEVHTAQTQTHREDYNS
TLRVVSALPIQHQDWMSGKEFKCKVNNKDLPAPIERTISKPKGSVRAPQ
VYVLPPPEEEMTKKQVTLTCMVTDFMPEDIYVEWTNNGKTELNYKNTEP
VLDSDGSYFMYSKLRVEKKNWVERNSYSCSVVHEGLHNHHTTKSFSRTP
GK
(HCVR of exemplified CNS-only expressed human tau
binding antibody)
SEQ ID NO: 17
QVQLQQWGAGLLKPSETLSLTCAVYGGSFSPYYWSWIRQPPDKGLEWIG
EINWSGDTNYNPSLKSRVTISLDTSKNQFSLNLSSVTAADTAVYYCARS
FDRWGQGTLVTVSS
(LC of exemplified CNS-only expressed human tau
binding antibody)
SEQ ID NO: 18
EIVLTQSPGTLSLSPGERATLSCRASQSVRSNYFAWYQQKPGQAPRLLI
YGVSRRAFGIPDRFSGSGSGTDFTLTISRLEPEDFAVYYCQQYGASLIT
FGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC
(LCVR of exemplified CNS-only expressed human tau
binding antibody)
SEQ ID NO: 19
EIVLTQSPGTLSLSPGERATLSCRASQSVRSNYFAWYQQKPGQAPRLLI
YGVSRRAFGIPDRFSGSGSGTDFTLTISRLEPEDFAVYYCQQYGASLIT
FGQGTRLEIK
(HCDR1 of exemplified CNS-only expressed human tau
binding antibody)
SEQ ID NO: 20
AVYGGSFSPYWS
(HCDR2 of exemplified CNS-only expressed human tau
binding antibody)
SEQ ID NO: 21
EINWSGDTN
(HCDR3 of exemplified CNS-only expressed human tau
binding antibody)
SEQ ID NO: 22
ARSFDR
(LCDR1 of exemplified CNS-only expressed human tau
binding antibody)
SEQ ID NO: 23
RASQSVRSNYFA
(LCDR2 of exemplified CNS-only expressed human tau
binding antibody)
SEQ ID NO: 24
YGVSRRAF
(LCDR3 of exemplified CNS-only expressed human tau
binding antibody)
SEQ ID NO: 25
QQYGASLIT
(exemplified peptide for immunization)
SEQ ID NO: 26
RTPSLPTPPTR
wherein T at residue 7 is phosphorylated
(exemplified peptide for immunization)
SEQ ID NO: 27
AEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPE
GTTAEEAGIGDTPSLEDEAAGHVTQEPESGKVVQEGFLREPGPPGLSHQ
LMSGMPGAPLLPEGPREATRQPSGTGPEDTEGGRHAPELLKHQLLGDLH
QEGPPLKGAGGKERPGSKEEVDEDRDVDESSPQDSPPSKASPAQDGRPP
QTAAREATSIPGFPAEGAIPLPVDFLSKVSTEIPASEPDGPSVGRAKGQ
DAPLEFTEHVEITPNVQKEQAHSEEHLGRAAFPGAPGEGPEARGPSLGE
DTKEADLPEPSEKQPAAAPRGKPVSRVPQLKARMVSKSKDGTGSDDKKA
KTSTRSSAKTLKNRPCLSPKHPTPGSSDPLIQPSSPAVCPEPPSSPKYV
SSVTSRTGSSGAKEMKLKGADGKTKIATPRGAAPPGQKGQANATRIPAK
TPPAPKTPPSSGEPPKSGDRSGYSSPGSPGTPGSRSRTPSLPTPPTREP
KKVAVVRTPPKSPSSAKSRLQTAPVPMPDLKNVKSKIGSTENLKHQPGG
GKVQIINKKLDLSNVQSKCGSKDNIKHVPGGGSVQIVYKPVDLSKVTSK
CGSLGNIFIRKPGGGQVEVKSEKLDFKDRVQSKIGSLDNITHVPGGGNK
KIETHKLTFRENAKAKTDHGAEIVYKSPVVSGDTSPRHLSNVSSTGSID
MVDSPQLATLADEVSASLAKQGL

We claim:

1. A method of detecting hTau-pT217 in a patient sample comprising the steps of:
contacting the patient sample with an antibody which specifically binds human tau phosphorylated at threonine at residue 217 of SEQ ID NO. 1 (“hTau-pT217”); and
detecting binding of the antibody with hTau-pT217.

2. The method of claim 1, wherein the antibody which specifically binds hTau-pT217 comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has an amino acid sequence with at least 95% homology to the amino acid sequence of SEQ ID NO: 13; LCDR2 has an amino acid sequence with at least 95% homology to the amino acid sequence of SEQ ID NO: 14, LCDR3 has an amino acid sequence with at least 95% homology to the amino acid sequence of SEQ ID NO: 15, HCDR1 has an amino acid sequence with at least 95% homology to the amino acid sequence of SEQ ID NO: 10, HCDR2 has an amino acid sequence with at least 95% homology to the amino acid sequence of SEQ ID NO: 11, and HCDR3 has an amino acid sequence with at least 95% homology to the amino acid sequence of SEQ ID NO: 12.

3. The method of claim 1, wherein the antibody which specifically binds hTau-pT217 comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino acid sequence of SEQ ID NO: 13; LCDR2 has the amino acid sequence of SEQ ID NO: 14, LCDR3 has the amino acid sequence of SEQ ID NO: 15, HCDR1 has the amino acid sequence of SEQ ID NO: 10, HCDR2 has the amino acid sequence of SEQ ID NO: 11, and HCDR3 has the amino acid sequence of SEQ ID NO: 12.

4. The method of claim 1, wherein the antibody which specifically binds hTau-pT217 comprises a light chain variable region (LCVR) and a heavy chain variable region (HCVR), wherein the LCVR and HCVR is selected from:
a. the LCVR having the amino acid sequence of SEQ ID NO: 5 and the HCVR having the amino acid sequence of SEQ ID NO: 3;
b. the LCVR having the amino acid sequence of SEQ ID NO: 8 and the HCVR having the amino acid sequence of SEQ ID NO: 6;
c. the LCVR having an amino acid sequence with at least 95% homology to the amino acid sequence of SEQ ID NO: 5 and the HCVR having an amino acid sequence with at least 95% homology to the amino acid sequence of SEQ ID NO: 3; and
d. the LCVR having an amino acid sequence with at least 95% homology to the amino acid sequence of SEQ ID NO: 8 and the HCVR having an amino acid sequence with at least 95% homology to the amino acid sequence of SEQ ID NO: 6.

5. The method of claim 1, wherein the antibody which specifically binds hTau-pT217 comprises a light chain (LC) and a heavy chain (HC), wherein the LC and the HC is selected from:
a. the LC having the amino acid sequence of SEQ ID NO: 4 and the HC having the amino acid sequence of SEQ ID NO: 2;
b. the LC having the amino acid sequence of SEQ ID NO: 9 and the HC having the amino acid sequence of SEQ ID NO: 7;
c. the LC having an amino acid sequence with at least 95% homology to the amino acid sequence of SEQ ID NO: 4 and the HC having an amino acid sequence with at least 95% homology to the amino acid sequence of SEQ ID NO: 2; and
d. the LC having an amino acid sequence with at least 95% homology to the amino acid sequence of SEQ ID NO: 9 and the HC having an amino acid sequence with at least 95% homology to the amino acid sequence of SEQ ID NO: 7.

6. The method of claim 1 further comprising the step of contacting the patient sample with a second antibody, said second antibody binding an epitope region of hTau-pT217 that does not overlap with the antibody,
wherein the second antibody specifically binds CNS-expressed isoforms of human tau.

7. The method of claim 6, wherein the second antibody binds an epitope region of human tau comprising glutamine at residue 124 and alanine at residue 125 of SEQ ID NO. 1.

8. The method of claim 6, wherein the second antibody comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has an amino acid sequence with at least 95% homology to the amino acid sequence of SEQ ID NO: 23; LCDR2 has an amino acid sequence with at least 95% homology to the amino acid sequence of SEQ ID NO: 24, LCDR3 has an amino acid sequence with at least 95% homology to the amino acid sequence of SEQ ID NO: 25, HCDR1 has an amino acid sequence with at least 95% homology to the amino acid sequence of SEQ ID NO: 20, HCDR2 has an amino acid sequence with at least 95% homology to the amino acid sequence of SEQ ID NO: 21, and HCDR3 has an amino acid sequence with at least 95% homology to the amino acid sequence of SEQ ID NO: 22.

9. The method of claim 6, wherein the second antibody comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino acid sequence of SEQ ID NO: 23; LCDR2 has the amino acid sequence of SEQ ID NO: 24, LCDR3 has the amino acid sequence of SEQ ID NO: 25, HCDR1 has the amino acid sequence of SEQ ID NO: 20, HCDR2 has the amino acid sequence of SEQ ID NO: 21, and HCDR3 has the amino acid sequence of SEQ ID NO: 22.

10. The method of claim 6, wherein the second antibody comprises a light chain variable region (LCVR) and a heavy chain variable region (HCVR), wherein the LCVR and HCVR is selected from:
a. the LCVR having the amino acid sequence of SEQ ID NO: 19 and the HCVR having the amino acid sequence of SEQ ID NO: 17; and
b. the LCVR having an amino acid sequence with at least 95% homology to the amino acid sequence of SEQ ID NO: 19 and the HCVR having an amino acid sequence with at least 95% homology to the amino acid sequence of SEQ ID NO: 17.

11. The method of claim 6, wherein the second antibody comprises a light chain (LC) and a heavy chain (HC), wherein the LC and the HC is selected from:
a. the LC having the amino acid sequence of SEQ ID NO: 18 and the HC having the amino acid sequence of SEQ ID NO: 16; and
b. the LC having an amino acid sequence with at least 95% homology to the amino acid sequence of SEQ ID NO: 18 and the HC having an amino acid sequence with at least 95% homology to the amino acid sequence of SEQ ID NO: 16.

12. The method of claim 6, wherein one of the antibody or the second antibody comprises a detectable label and said step of detecting comprises detecting a signal provided by the detectable label upon formation of a complex comprising the antibody, the second antibody and hTau-pT217.

13. The method of claim 1, further comprising the step of quantifying hTau-pT217 in the patient sample.

14. The method of claim 1, wherein the patient sample is one of blood, plasma, serum or CSF.

15. A method of treating a neurodegenerative disease in a patient in need of thereof comprising the steps of:
contacting a sample from the patient with an antibody which specifically binds human tau phosphorylated at threonine at residue 217 of SEQ ID NO. 1 (“hTau-pT217”);
detecting binding of the antibody with hTau-pT217 in the sample from the patient; and
administering to the patient a therapeutically effective amount of a therapeutic antibody that specifically binds amyloid beta or tau.

16. The method of claim 15, wherein the antibody which specifically binds hTau-pTau217 comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has an amino acid sequence with at least 95% homology to the amino acid sequence of SEQ ID NO: 13; LCDR2 has an amino acid sequence with at least 95% homology to the amino acid sequence of SEQ ID NO: 14, LCDR3 has an amino acid sequence with at least 95% homology to the amino acid sequence of SEQ ID NO: 15, HCDR1 has an amino acid sequence with at least 95% homology to the amino acid sequence of SEQ ID NO: 10, HCDR2 has an amino acid sequence with at least 95% homology to the amino acid sequence of SEQ ID NO: 11, and HCDR3 has an amino acid sequence with at least 95% homology to the amino acid sequence of SEQ ID NO: 12.

17. The method of claim 15, wherein the neurodegenerative disease is Alzheimer's disease (AD).

18. A method of diagnosing a patient as one or more of: (i) having a neurodegenerative disease; (ii) at risk for having a neurodegenerative disease; (iii) in need of treatment for a neurodegenerative disease; or (iv) in need of neurological imaging comprising the steps of:
contacting a sample from the patient sample with an antibody which specifically binds hTau-p217;
contacting the patient sample with a second antibody that specifically binds CNS-expressed isoforms of human tau; and
detecting binding between the antibody and hTau-pT217 in the patient sample,
wherein the second antibody binds an epitope region of human tau comprising glutamine at residue 124 and alanine at residue 125 of SEQ ID NO. 1.

19. The method of claim 18, wherein the antibody which specifically binds hTau-pTau217 comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has an amino acid sequence with at least 95% homology to the amino acid sequence of SEQ ID NO: 13; LCDR2 has an amino acid sequence with at least 95% homology to the amino acid sequence of SEQ ID NO: 14, LCDR3 has an amino acid sequence with at least 95% homology to the amino acid sequence of SEQ ID NO: 15, HCDR1 has an amino acid sequence with at least 95% homology to the amino acid sequence of SEQ ID NO: 10, HCDR2 has an amino acid sequence with at least 95% homology to the amino acid sequence of SEQ ID NO: 11, and HCDR3 has an amino acid sequence with at least 95% homology to the amino acid sequence of SEQ ID NO: 12.

20. The method of claim 18, wherein the second antibody comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has an amino acid sequence with at least 95% homology to the amino acid sequence of SEQ ID NO: 23; LCDR2 has an amino acid sequence with at least 95% homology to the amino acid sequence of SEQ ID NO: 24, LCDR3 has an amino acid sequence with at least 95% homology to the amino acid sequence of SEQ ID NO: 25, HCDR1 has an amino acid sequence with at least 95% homology to the amino acid sequence of SEQ ID NO: 20, HCDR2 has an amino acid sequence with at least 95% homology to the amino acid sequence of SEQ ID NO: 21, and HCDR3 has an amino acid sequence with at least 95% homology to the amino acid sequence of SEQ ID NO: 22.


SEQUENCE LISTING (27 sequences)

SEQ ID NO: 1 - protein, 441 aa, Artificial Sequence
  Synthetic Sequence
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 433 aa, Artificial Sequence
  Synthetic construct
QSVEESGGRLVTPGTPLTLTCTVSGLSPSWYGVHWVRQAPGKGLEWIGVLRAGSHTYYAGWAKGRFAISKTSTTVALKITSPTTEDTAIYFCGSVGRGIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 3 - protein, 110 aa, Artificial Sequence
  Synthetic Sequence
QSVEESGGRLVTPGTPLTLTCTVSGLSPSWYGVHWVRQAPGKGLEWIGVLRAGSHTYYAGWAKGRFAISKTSTTVALKITSPTTEDTAIYFCGSVGRGIWGPGTLVTVSL

SEQ ID NO: 4 - protein, 217 aa, Artificial Sequence
  Synthetic Sequence
AQVLTQTASPVSATVGGTVTINCQASLAVYNNNYLAWYQQKPGQPPKRLIYLASSLSSGVSSHFKGSGSGTQFTLTISDVQADDAATYFCQGSYDCTIADCVAFGGGTEVVVKGDPVAPTVLIFPPAADQVATGTVTIVCVANKYFPDVTVTWEVDGTTQTTGIENSKTPQNSADNTYNLSSTLTLTSTQYNSHKEYTCKVTQGTTSVVQSFNRGDC

SEQ ID NO: 5 - protein, 113 aa, Artificial Sequence
  Synthetic Sequence
AQVLTQTASPVSATVGGTVTINCQASLAVYNNNYLAWYQQKPGQPPKRLIYLASSLSSGVSSHFKGSGSGTQFTLTISDVQADDAATYFCQGSYDCTIADCVAFGGGTEVVVK

SEQ ID NO: 6 - protein, 110 aa, Artificial Sequence; identical to SEQ ID NO: 3
  Synthetic Sequence

SEQ ID NO: 7 - protein, 434 aa, Artificial Sequence
  Synthetic Sequence
QSVEESGGRLVTPGTPLTLTCTVSGLSPSWYGVHWVRQAPGKGLEWIGVLRAGSHTYYAGWAKGRFAISKTSTTVALKITSPTTEDTAIYFCGSVGRGIWGPGTLVTVSLAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 113 aa, Artificial Sequence; identical to SEQ ID NO: 5
  Synthetic Sequence

SEQ ID NO: 9 - protein, 220 aa, Artificial Sequence
  Synthetic Sequence
AQVLTQTASPVSATVGGTVTINCQASLAVYNNNYLAWYQQKPGQPPKRLIYLASSLSSGVSSHFKGSGSGTQFTLTISDVQADDAATYFCQGSYDCTIADCVAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 10 aa, Artificial Sequence
  Synthetic Sequence
GLSPSWYGVH

SEQ ID NO: 11 - protein, 16 aa, Artificial Sequence
  Synthetic Sequence
VLRAGSHTYYAGWAKG

SEQ ID NO: 12 - protein, 5 aa, Artificial Sequence
  Synthetic Sequence
VGRGI

SEQ ID NO: 13 - protein, 13 aa, Artificial Sequence
  Synthetic Sequence
QASLAVYNNNYLA

SEQ ID NO: 14 - protein, 7 aa, Artificial Sequence
  Synthetic Sequence
LASSLSS

SEQ ID NO: 15 - protein, 7 aa, Artificial Sequence
  Synthetic Sequence
LASSLSS

SEQ ID NO: 16 - protein, 443 aa, Artificial Sequence
  Synthetic Sequence
QVQLQQWGAGLLKPSETLSLTCAVYGGSFSPYYWSWIRQPPDKGLEWIGEINWSGDTNYNPSLKSRVTISLDTSKNQFSLNLSSVTAADTAVYYCARSFDRWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 112 aa, Artificial Sequence
  Synthetic Sequence
QVQLQQWGAGLLKPSETLSLTCAVYGGSFSPYYWSWIRQPPDKGLEWIGEINWSGDTNYNPSLKSRVTISLDTSKNQFSLNLSSVTAADTAVYYCARSFDRWGQGTLVTVSS

SEQ ID NO: 18 - protein, 215 aa, Artificial Sequence
  Synthetic Sequence
EIVLTQSPGTLSLSPGERATLSCRASQSVRSNYFAWYQQKPGQAPRLLIYGVSRRAFGIPDRFSGSGSGTDFTLTISRLEPEDFAVYYCQQYGASLITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 108 aa, Artificial Sequence
  Synthetic Sequence
EIVLTQSPGTLSLSPGERATLSCRASQSVRSNYFAWYQQKPGQAPRLLIYGVSRRAFGIPDRFSGSGSGTDFTLTISRLEPEDFAVYYCQQYGASLITFGQGTRLEIK

SEQ ID NO: 20 - protein, 13 aa, Artificial Sequence
  Synthetic Sequence
AVYGGSFSPYYWS

SEQ ID NO: 21 - protein, 9 aa, Artificial Sequence
  Synthetic Sequence
EINWSGDTN

SEQ ID NO: 22 - protein, 6 aa, Artificial Sequence
  Synthetic Sequence
ARSFDR

SEQ ID NO: 23 - protein, 12 aa, Artificial Sequence
  Synthetic Sequence
RASQSVRSNYFA

SEQ ID NO: 24 - protein, 8 aa, Artificial Sequence
  Synthetic Sequence
YGVSRRAF

SEQ ID NO: 25 - protein, 9 aa, Artificial Sequence
  Synthetic Sequence
QQYGASLIT

SEQ ID NO: 26 - protein, 11 aa, Artificial Sequence
  Synthetic Sequence
  MISC_FEATURE (7)..(7): wherein T at residue 7 is phosphorylated
RTPSLPTPPTR

SEQ ID NO: 27 - protein, 757 aa, Artificial Sequence
  Synthetic Sequence
AEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL